(19) United States
(12) Patent Application Publication
(10) Pub. No.: US 2018/0199954 A1
(43) Pub. Date: Jul. 19, 2018

(54) SEALING AND STABILIZING DEVICE FOR USE WITH ENDOSCOPIC SHAVERS

(71) Applicant: Tenjin LLC, Brazoria, TX (US)

(72) Inventors: Christopher P. DOUGHERTY, Rogers, AR (US); Gary R. Heisler, Brazoria, TX (US); Robert A. Van Wyk, St. Pete Beach, FL (US)

(73) Assignee: Tenjin LLC, Brazoria, TX (US)

(21) Appl. No.: 15/873,749

(22) Filed: Jan. 17, 2018

Related U.S. Application Data
(60) Provisional application No. 62/499,155, filed on Jan. 17, 2017.

(51) Int. Cl. A61B 17/32; A61B 17/16

(52) U.S. Cl. CPC A61B 17/32002; A61B 2017/3441; A61B 17/1613

(57) ABSTRACT

Described herein is combination sealing and stabilizing collar component designed to both prevent the leakage of vacuum through the interface between an endoscopic shaver device and a powered handpiece that provides rotational motion thereto and stabilize the endoscopic shaver device when it is mounted onto the powered handpiece. The collar may be further coupled with a conventional cannula to prevent the leakage of intra-articular fluid when performing an arthroscopic procedure.

### PRIORITY

This application claims the benefit of U.S. Provisional Application Ser. No. 62/499,155 filed Jan. 17, 2017, the contents of which are hereby incorporated by reference in its entirety.

### TECHNICAL FIELD OF THE INVENTION

The present invention relates generally to the field of endoscopic and arthroscopic surgery, more particularly to a sealing and stabilizing devices for use with powered endoscopic shaver assemblies designed for such surgeries. In a particularly preferred embodiment, the present invention relates to a collar component of an endoscopic shaver assembly that is designed to that both prevent the leakage of vacuum through the interface between an endoscopic shaver device and a powered handpiece that provides rotational motion thereto and stabilizes the endoscopic shaver device when it is mounted onto the powered handpiece. The collar component may additionally be coupled to a conventional cannula to prevent the leakage of intra-articular fluid when performing an arthroscopic procedure.

### BACKGROUND OF THE INVENTION

Endoscopic shavers are well known in the art. The construction and use of illustrative examples of such a device is described in U.S. Pat. No. 4,203,444 to Bonnell et al. and U.S. Pat. No. 4,646,738 to Trott, as well as U.S. Pat. Nos. 7,66,200, 8,313,502 and 9,486,232 to Heisler et al., among others.

Endoscopic shavers (also referred to as “arthroscopic” shavers) may be designed for single-use or as reusable devices that may be mounted onto a reusable handpiece that powers the shaver blades. A typical handpiece is configured to provide suction to the shaver along with rotational motion. Critically, adequate clearance must be provided between the mounting element (often referred to as an “outer hub”) of the shaver and the accepting orifice of the handpiece. The gap afforded by this clearance can provide a path for vacuum leakage. As such leakage decreases the vacuum applied to the shaver and thereby reduces its efficiency, there is strong desire in the arts to minimize and/or avoid it entirely. Additionally, this gap can cause the shaver to be inadequately mounted in the handpiece and thus allow the inner member of the shaver device to deflect from the preferred linear configuration or otherwise move around during use. Accordingly, there is a clear need in the art to strengthen and securely fix the connection between the shaver device and the handpiece. Thus, a primary objective of the present invention is to resolve the problems of leakage and deflection and thereby provide a more efficient, effective endoscopic shaver assembly.

### SUMMARY OF THE INVENTION

The conventional endoscopic shaver assembly is comprised of an elongated inner member axially slidable and rotatably situated within an elongated stationary outer member, wherein both inner and outer members have coordinating hubs at their respective proximal ends and cutting apertures which cooperate to resect tissue during endoscopic surgical procedures at their respective distal ends. The distal end of the outer hub of a shaver blade typically incorporates a means for removably mounting a cannula thereto. For instance, the Universal Cannula products by Conmed, Incorporated (Utica, N.Y.) may be mounted to Conmed's Ultra and Sterling shaver blades, along with others of their manufacture. The mounting means is generally comprised of complementary internal or external threaded portions on the cannula and outer hub. Collar devices of the present invention removably mount to the distal portion of the outer hub of a shaver blade in the manner of prior art cannulas. However, unlike prior art cannulas, preferred embodiments of the present invention have a flange that blocks flow through the gap between the shaver's outer hub and the shaver handpiece, thereby increasing the efficiency of the shaver by decreasing the vacuum loss through the gap. Additionally, the collar components of the present invention stabilize the shaver so as to reduce deflection of the shaver distal portion during use.

Thus, in one preferred embodiment, the present invention provides a combination sealing and stabilizing device adapted for use with an endoscopic shaver that includes:
a. an elongate tubular outer member characterized by a distal end having an outer cutting aperture formed therein, a proximal end that includes an outer hub configured for removable mounting within a cavity of an endoscopic shaver handpiece, and an elongate central lumen extending between the proximal and distal ends that defines the longitudinal axis of the shaver assembly, wherein the distal portion of said outer hub includes a first set of threads, further wherein distal axial movement of the outer member relative to the endoscopic shaver handpiece is precluded by means of an annular locking collet disposed about the outer periphery of the outer hub, further wherein the annular locking collet defines a gap between the outer periphery of the outer hub and an inner periphery of the collet; and
b. an elongate tubular inner member characterized by a distal end having an inner cutting aperture formed therein, a proximal end that includes an inner hub configured for receiving torque from the endoscopic shaver handpiece and a proximal port for transmitting suction from the endoscopic shaver handpiece to the inner cutting aperture via an elongate central lumen that extends between the proximal and distal ends of the inner member, wherein the inner member is slidably received within the central lumen of the outer member and axially extended such that the inner and outer cutting apertures align.

In a particularly preferred embodiment, the combination sealing and stabilizing device takes the form of a collar having a proximal end, a distal end, a central opening extending between the proximal and distal ends, a circumferential rim extending radially from the central opening characterized by proximally and distal facing surfaces, and a second set of threads complementary to and configured to engage with the first said of threads on the outer hub, wherein the combination sealing and stabilizing collar is mounted over the elongate outer member and positioned against the distal end of the outer hub, further wherein engaging the first and second sets of threads causes the proximal facing surface of the circumferential rim to be firmly seated against the annular locking collet and thereby seal the gap disposed between the outer hub and the annular locking collet and preclude lateral movement of outer member.

The sealing and stabilizing collar, the endoscopic shaver, and the shaver handpiece may take the form of a kit or assembly that may optionally further include a cannulated access device such as conventional in the art.

These and other aspects are accomplished in the invention herein described, directed to sealing and stabilizing mechanisms for use with powered endoscopic shaver assemblies. Further objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention.

### BRIEF DESCRIPTION OF THE FIGURES

Various aspects and applications of the present invention will become apparent to the skilled artisan upon consideration of the brief description of figures and the detailed description of the present invention and its preferred embodiments that follows:

FIG. 1 depicts a prior art shaver blade with the inner assembly removed from the outer assembly.

FIG. 2 is a side elevational view of the prior art shaver of FIG. 1 with the shaver assembled for use.

FIG. 3 is a perspective view of the objects of FIG. 2.

FIG. 4 is an expanded view of the objects of FIG. 3 at location A.

FIG. 5 is a plan view of the objects of FIG. 2.

FIG. 6 is an expanded sectional view of the objects of FIG. 5 at location A-A.

FIG. 7 is a perspective view of the prior art shaver of FIG. 2 in a prior art endoscopic shaver handpiece in preparation for use.

FIG. 8 is an expanded view of the objects of FIG. 7 at location A.

FIG. 9A is a plan view of the objects of FIG. 7.

FIG. 9B is a side elevational view of the objects of FIG. 9A.

FIG. 10 is an expanded sectional view of the objects of FIG. 9A at location A-A.

FIG. 11 is a distal perspective view of a sealing/stabilizing collar component of the present invention.

FIG. 12 is a proximal perspective view of a sealing/stabilizing collar component of the present invention.

FIG. 13 is a distal axial view of the objects of FIG. 11.

FIG. 14 is a side elevational view of the objects of FIG. 11.

FIG. 15 is a proximal axial view of the objects of FIG. 11.

FIG. 16 is a perspective view of a sealing/stabilizing collar component of the present invention mounted on a prior art shaver and endoscopic shaver handpiece in preparation for use.

FIG. 17 is an expanded view of the objects of FIG. 16 at location A.

FIG. 18 is a plan view of the objects of FIG. 16.

FIG. 19 is an expanded sectional view of the objects of FIG. 18 at location A-A.

FIG. 20 is a proximal perspective exploded assembly view of an alternate embodiment sealing/stabilizing collar component of the present invention.

FIG. 21 is a distal perspective exploded assembly view of the objects of FIG. 20.

FIG. 22 is a proximal perspective view of the alternate embodiment sealing/stabilizing collar component of FIG. 20, assembled.

FIG. 23 is a side elevational view of the objects of FIG. 22.

FIG. 24 is a plan view of the objects of FIG. 22.

FIG. 25A is a sectional view of the objects of FIG. 24 at location A-A.

FIG. 25B is an expanded view of the objects of FIG. 25A at location A.

FIG. 26 is a perspective view of an alternate embodiment sealing/stabilizing collar component of the present invention mounted on a prior art shaver and endoscopic shaver handpiece in preparation for use.

FIG. 27 is a side elevational view of an alternate prior art shaver mounted in a prior art endoscopic shaver handpiece in preparation for use.

FIG. 28 is an expanded view of the objects of FIG. 27 at location A.

FIG. 29 is a perspective view of the objects of FIG. 27.

FIG. 30 is an expanded view of the objects of FIG. 29 at location B.

FIG. 31 is a plan view of an alternate embodiment sealing/stabilizing collar component of the present invention.

FIG. 32 is a proximal perspective view of the objects of FIG. 31.

FIG. 33 is a distal perspective view of the objects of FIG. 31.

FIG. 34 is a sectional view of the objects of FIG. 31 at location A-A.

FIG. 35 is a side elevational view of the alternate embodiment sealing/stabilizing collar component of FIG. 31 mounted to the alternate prior art shaver mounted and prior art endoscopic shaver handpiece of FIG. 27 in preparation for use.

FIG. 36 is an expanded view of the objects of FIG. 35 at location A.

FIG. 37 is a perspective view of the objects of FIG. 35.

FIG. 38 is an expanded view of the objects of FIG. 37 at location B.

### DETAILED DESCRIPTION OF THE PREFERRED EMBODIMENTS

Aspects of the present invention relate to, overlap with and/or find utility in conjunction with aspects described in the following related patents and patent applications, the entire contents of which are hereby incorporated in their entirety:
U.S. Pat. No. 9,486,232 issued Jan. 1, 2016 to Heisler et al.;
U.S. Pat. No. 7,666,200 issued Feb. 23, 2010; U.S. Pat. No. 8,088,135 issued Jan. 3, 2012; U.S. Pat. No. 8,177,803 issued May 15, 2012; and U.S. Pat. No. 8,313,502 issued Nov. 20, 2012 to Heisler;
U.S. application Ser. No. 14/470,422 filed Aug. 27, 2014 and published as US 2015/0065808 to Van Wyk et al.; and
U.S. application Ser. No. 15/288,509 filed Oct. 7, 2016 and published as US 2017/100136 to Dougherty et al.
   Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Accordingly, unless otherwise defined, all technical and scientific terms used herein have the same meaning as commonly understood by one of ordinary skill in the art to which the present invention belongs. However, in case of conflict, the present specification, including definitions below, will control.

In the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

The term “proximal” as used herein refers to that end or portion which is situated closest to the user of the device, farthest away from the target surgical site. In the context of the present invention, the proximal face of the collar device of the present invention configured for use with an endoscopic shaver assembly may include one or more threaded components for mounting the collar to the distal end of an outer hub component and providing a fluid tight seal to the aspiration lumen therewithin.

The term “distal” as used herein refers to that end or portion situated farthest away from the user of the device, closest to the target surgical site. In the context of the present invention, the distal face of the collar of the present invention is configured to coordinate with a valved introduction cannula.

In the context of the present invention, the terms “cannula” and “cannulated” are used to generically refer to the family of rigid or flexible, typically elongate lumened surgical instruments that facilitate access across tissue to an internally located surgery site.

The terms “tube” and “tubular” are interchangeably used herein to refer to a generally round, long, hollow component having at least one central opening often referred to as a “lumen”.

The terms “lengthwise” and “axial” as used interchangeably herein to refer to the direction relating to or parallel with the longitudinal axis of a device. The term “transverse” as used herein refers to the direction lying or extending across or perpendicular to the longitudinal axis of a device.

The term “rotational” as used herein refers to the revolutionary movement about the center point or longitudinal axis of the device. In the context of the present invention, rotation of the elongated inner tubular member relative to the elongated outer tubular member, which typically is held in a stationary position, results in relative rotation of their respective cutting apertures which coordinate to resect target tissue within the surgical site of interest.

The term “lateral” pertains to the side and, as used herein, refers to motion, movement, or materials that are situated at, proceeding from, or directed to a side of a device.

The term “medial” pertains to the middle, and as used herein, refers to motion, movement or materials that are situated in the middle, in particular situated near the median plane or the midline of the device or subset component thereof.

The term “radial” is used herein to refer to characterize movement inward and outward from a central point or shaft, e.g., thrusts radial to the center of rotation.

The present invention contemplates the use of alternative cooperating elements, in particular cooperating elements of the collar device and the outer hub of the endoscopic shaver that coordinate to ensure fluid tight attachment of the collar to the endoscopic shaver assembly. While the invention is described herein below with respect to mating screw threads, other cooperating elements are contemplated, examples of which include, but are not limited to, worm gears, worm wheels, pneumatic devices, hydraulic mechanisms, magnetic assemblies, ratchet-and-pawl assemblies, and push-pull connectors.

In the context of the present invention, reference is made to various lock-and-key type mating mechanisms that serve to establish and secure the axial and rotational arrangement of various concentric or relatively slidable device components. It will be readily understood by the skilled artisan that the position of the respective coordinating elements (e.g., recessed slots and grooves that mate with assorted projecting protrusions, protuberances, tabs and splines) may be exchanged and/or reversed as needed.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal, more preferably a human.

Hereinafter, the present invention is described in more detail by reference to the Figures and Examples. However, the following materials, methods, figures, and examples only illustrate aspects of the invention and are in no way intended to limit the scope of the present invention. For example, while the present invention makes specific reference to arthroscopic procedures, it is readily apparent that the teachings of the present invention may be applied to other minimally invasive procedures and thus are not limited to arthroscopic uses alone. As such, methods and materials similar or equivalent to those described herein can be used in the practice or testing of the present invention.

### EXAMPLES

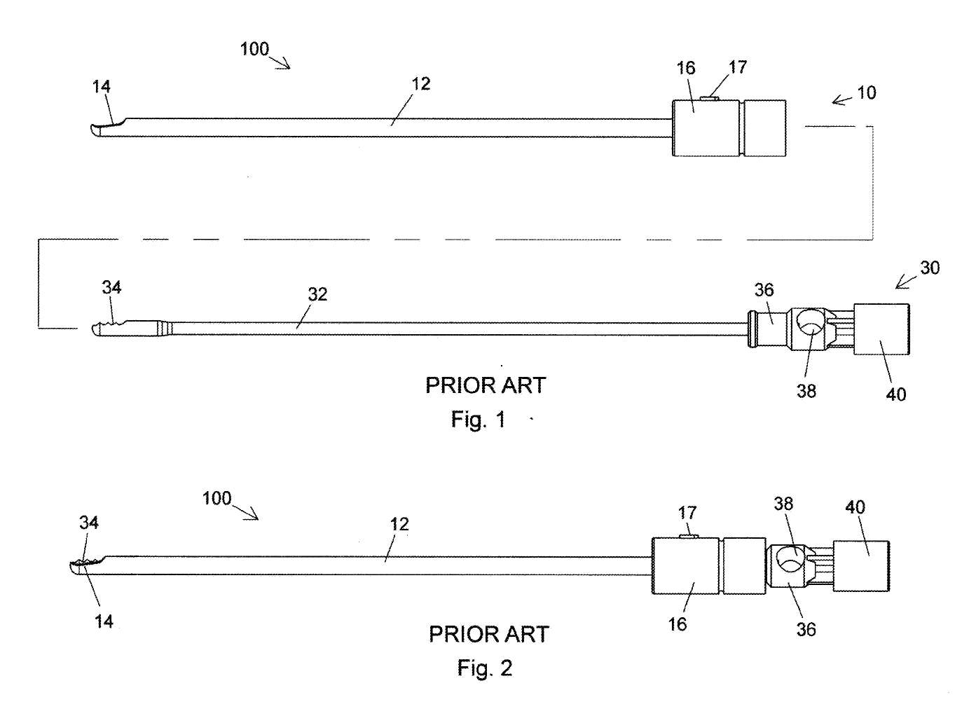

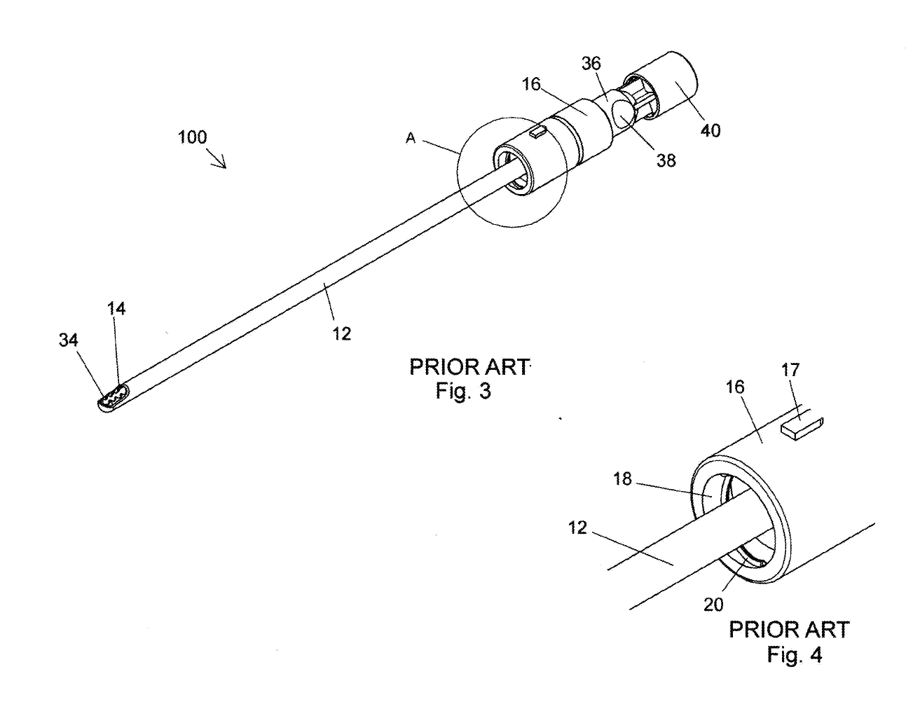

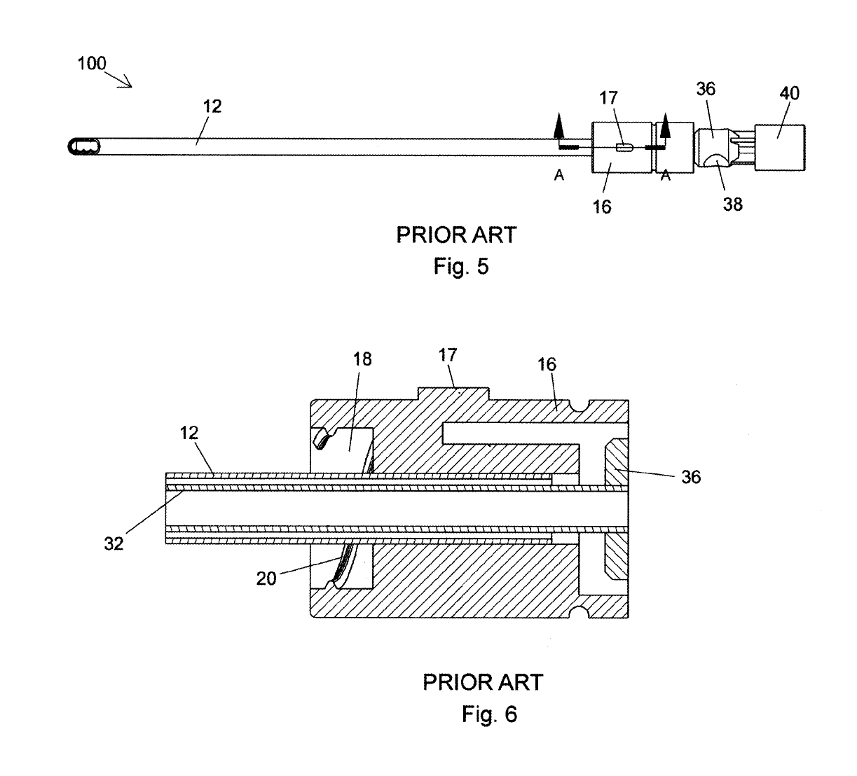

Referring to FIG. 1, prior art endoscopic shaver 100 has an outer assembly 10 with an elongate tubular member 12 with an aperture 14 formed at its distal end and, at its proximal end, an outer hub 16 configured for removable mounting in an endoscopic shaver handpiece. Shaver 100 also has an inner assembly 30 with an elongate tubular member 32 with an aperture 34 formed at its distal end, and, at its proximal end, a hub 36 configured for receiving torque from an endoscopic shaver handpiece when removably mounted therein, and for transmitting suction from the handpiece via port 38 formed in hub 36 and tubular member 32 to aperture 34. FIGS. 2 through 6 depict shaver 100 assembled for use. Outer hub 16 has formed in its distal end a cylindrical recess 18 wherein are formed threads 20. In use, inner assembly 30 is rotated within outer assembly 10. When aperture 14 of outer assembly 10 aligns with aperture 34 of inner assembly 30, suction supplied via inner tubular member 32 draws tissue into the aligned apertures wherein. As the apertures rotate past each other, tissue trapped between the approaching lateral edges of the inner aperture 14 and outer aperture 34 is cut and aspirated from the site, with inner tubular member 32, port 38 of inner hub 36, and means within the shaver handpiece forming an aspiration path to an external receptacle.

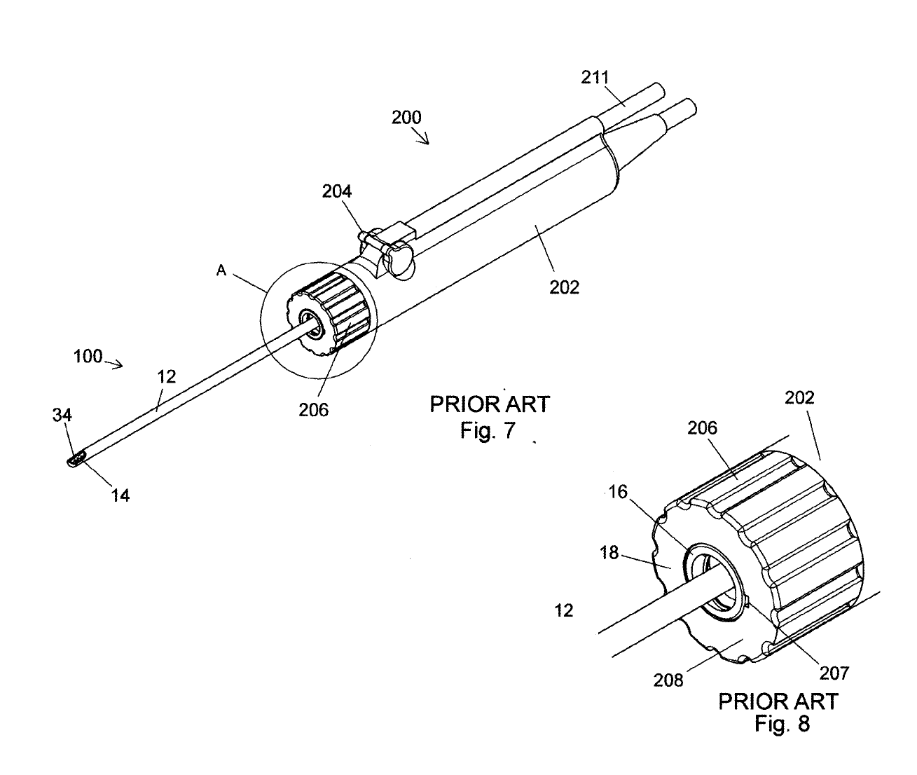

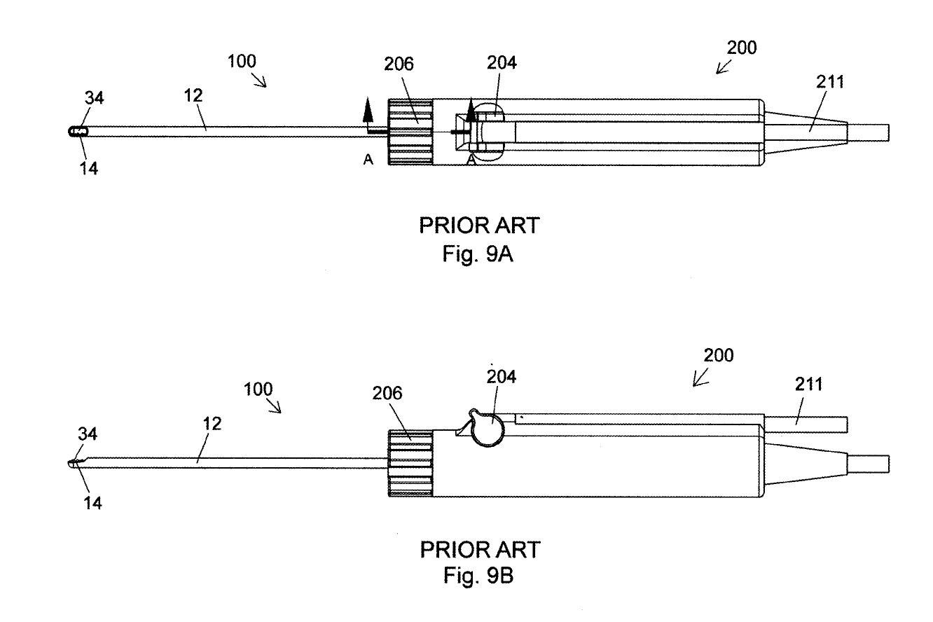

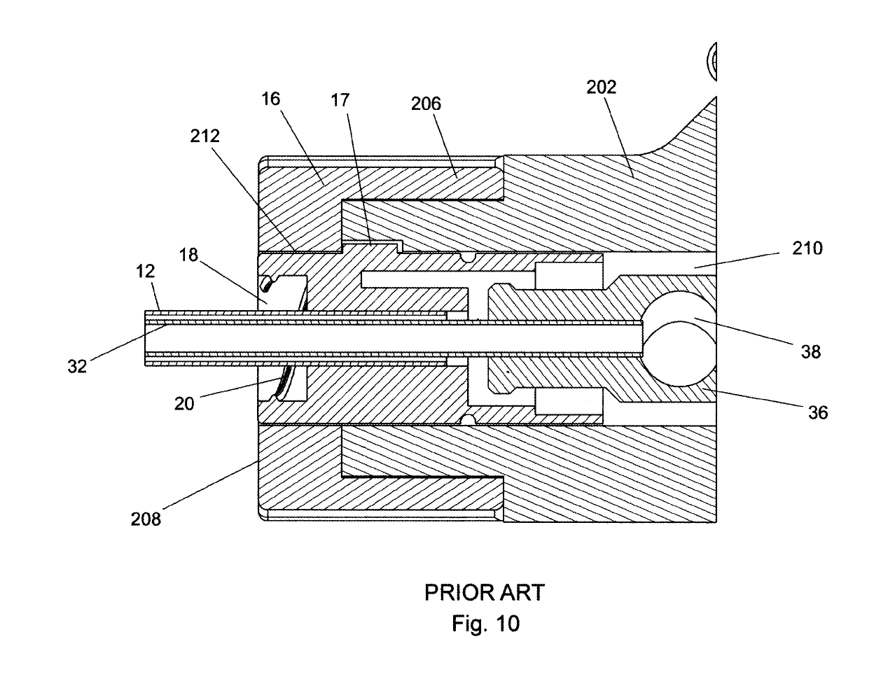

FIGS. 7 through 10 depict prior art shaver 100 removably mounted to prior art endoscopic shaver handpiece 200, wherein outer hub 16 is retained in the mounted position by collet 206 of handpiece 200. Collet 206 is rotatable between a first position, in which key 17 of outer hub 16 is aligned with keyway 207 of collet 206 so as to allow shaver 100 to be inserted into handpiece 200, and a second position (see FIG. 10), in which collet 206 prevents demounting of shaver 100 from handpiece 200 by preventing distal movement of outer hub 16. In use, suction supplied by tubular member 211 of handpiece 200 is conducted by means within handpiece body 202 to cavity 210 (see FIG. 10) wherein is positioned inner hub 36. The vacuum within cavity 210 is transmitted via port 38 in inner hub 36 and the lumen of tubular member 32 to distal aperture 34. In use, tissue removed by shaver 100 is conducted via the lumen of tubular member 32, aperture 38 in inner hub 36, cavity 210, and means within handpiece body 202 to tubular member 211, and therefrom to an external collection cannister (not shown). Aspiration control 204 has a first position in which the vacuum path in handpiece body 202 is blocked, and a second position in which vacuum is conducted to cavity 210 such that tissue and fluid is aspirated from the surgical site.

Tissue is resected by shaver 100 in the following manner:

Inner assembly 30 of endoscopic shaver 100 is rotatably positioned within outer assembly 10. Handpiece 200 imparts a rotational (generally oscillating) motion to inner assembly 30 through inner hub 36 which engages with a drive mechanism (now shown) within shaver handpiece body 202. When aperture 34 of inner assembly 30 comes into alignment with aperture 14 of outer assembly 10, tissue is drawn into the aligned apertures by suction supplied thereto as previously described. As aperture 34 rotates past aperture 10, tissue trapped between lateral edges of the apertures is resected, the edges of apertures 34 and 10 being configured for that purpose. The resected tissue is aspirated from the site in the manner previously herein described. The efficiency of shaver 100 is strongly affected by the level of vacuum which draws tissue into aligned apertures 14 and 34. A decrease in the vacuum at the apertures results in less tissue being drawn into the aligned apertures and therefore less tissue resection.

Referring again to FIG. 10, cavity 210 to which suction is supplied is closed at its distal end by outer hub 16 of outer assembly 10. There is, however, a gap 212 formed between collet 206 of handpiece 200 and outer hub 16, and between the wall of cavity 210 of handpiece body 202 and outer hub 16. Leakage at the proximal end, through gap 212, decreases the vacuum level applied to the distal end, namely aperture 34 of inner assembly 30, which, in turn, results in a decrease in the resection rate. The size of gap 212 is determined by the repeatability of the manufacturing processes used to make collet 206 and body 202 of handpiece 200, and the molding process used to form polymeric outer hub 16 of shaver 100. While collet 206 and handpiece body are formed using highly accurate and reproducible processes, the accuracy of the injection molding process used to form outer hub 16 is strongly dependent on the degree to which the molding parameters are controlled. If the molder who makes outer hub 16 maintains close control of the molding parameters, the repeatability of the process will be high, thereby producing hubs 16 with a high degree of accuracy and only small variations in size. Because the minimum specified size of gap 212 is principally determined by the degree of variation in the size of hub 16, maintaining close control of the molding parameters when producing outer hub 16 is desirable. However, maintaining close control of molding parameters increases the cost of outer hub 16. To save money, some manufacturers of endoscopic shaver systems maintain less control of the molding parameters, thereby allowing for increased variation in the size of outer hub 16. To accommodate this increased variation, the associated gap 212 is increased. This, however, increases vacuum loss through gap 212 and decreases the efficiency of shaver 100.

In an attempt to minimize vacuum leakage through gap 212, various sealing methods have been contemplated by the prior art. For example, in some cases an elastomeric seal is incorporated in handpiece body 202 or collet 206. Because handpiece 200 is typically sterilized in an autoclave between uses, polymeric seals that are part of handpiece 200 may, over time, lose their elasticity and become ineffective at preventing vacuum leakage. In other cases, an elastomeric seal is positioned on outer hub 16. In such cases, the effectiveness of the seal is affected by the size of hub 16 and thus the effectiveness of the seal varies accordingly.

In addition to contributing to vacuum loss, gap 212 also results in shaver 100 not rigidly being held in handpiece 200. For example, while the axial position of shaver 100 is determined by collet 206, the distal end of shaver 100 may deflect from its ideal axial position during use, with the degree of deflection being determined by the size of gap 212. This deflection may cause the distal end of shaver 100 to wobble during use.

The present invention addresses the problems of leakage and deflection by providing a combination sealing and stabilizing collar component that prevents vacuum leakage through gap 212, and makes rigid the fixation of shaver 100 in handpiece 200.

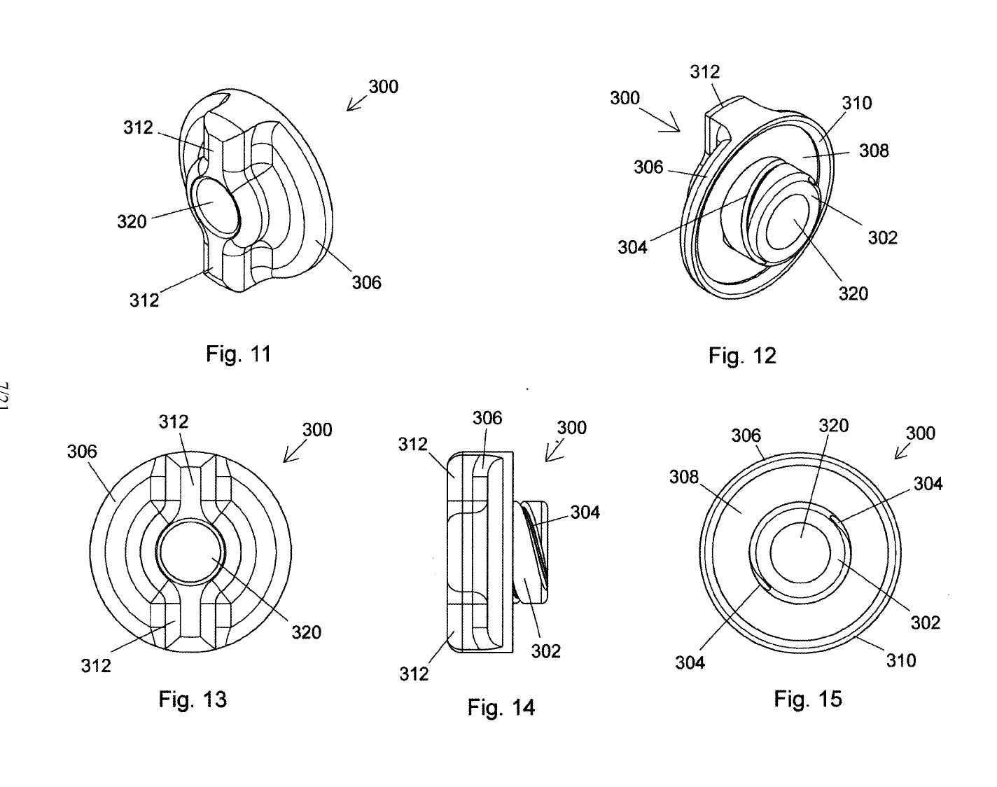

In a first illustrative embodiment depicted in FIGS. 11 through 15, sealing/stabilizing collar 300 has a cylindrical proximal portion 302 configured to be received within distal cylindrical recess 18 of outer hub 16. Threads 304 formed in cylindrical proximal portion 302 of sealing/stabilizing collar 300 are configured for coupling with threads 20 of cylindrical distal recess 18 of outer hub 16 such that sealing/stabilizing collar 300 may be removably mounted to outer hub 16 by threading of proximal portion 302 of collar 300 into distal recess 18 of outer hub 16. Sealing/stabilizing collar 300 has a distal portion 306 on which are formed distally extending flanges 312 for the purpose facilitating the threading of proximal portion 302 into cylindrical distal recess 18 of outer hub 16. Sealing/stabilizing collar component 300 has a central lumen 320 and a proximally extending rim 310 formed on proximal surface 308.

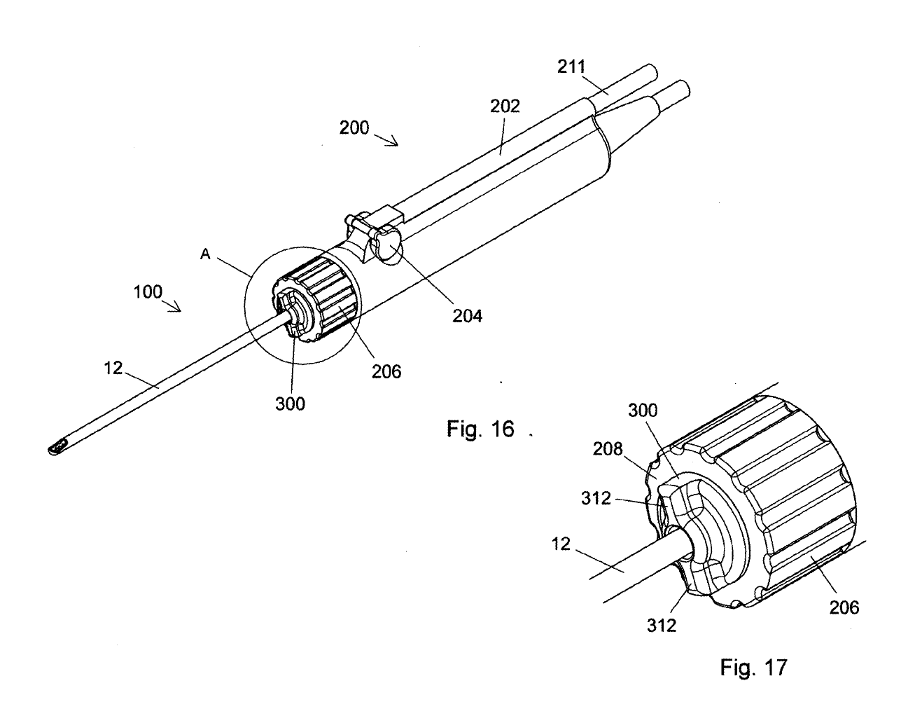

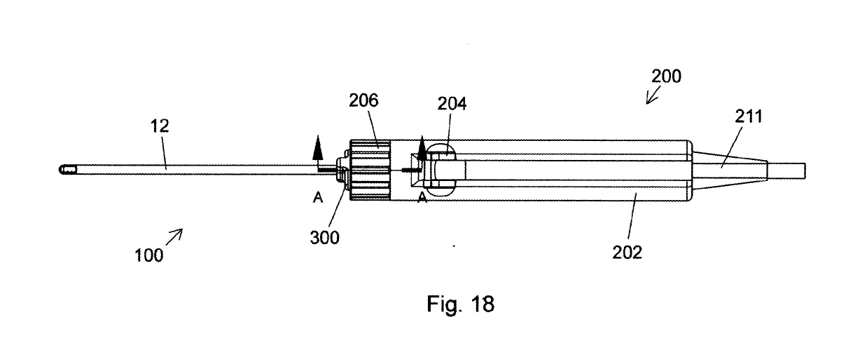

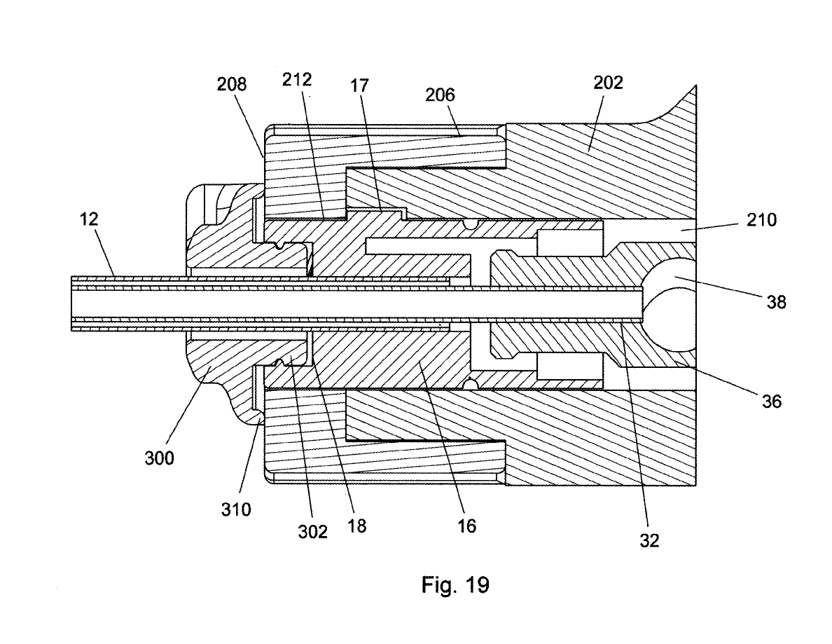

FIGS. 16 through 19 depict sealing/stabilizing collar 300 removably mounted to shaver 100 in accordance with the principles of the present invention. Proximal portion 302 of sealing/stabilizing collar 300 is threaded into recess 18 of outer hub 16 until proximally extending rim 310 of sealing/stabilizing collar 300 is firmly seated against distal-most surface 208 of annular collet 206 of handpiece 200. As best seen in FIG. 19, the path for vacuum leakage through gap 212 is blocked by sealing/stabilizing collar 300, and lateral movement of shaver 100 is prevented by contact between proximal rim 310 of sealing/stabilizing collar 300 and distal-facing surface 208 of collet 206.

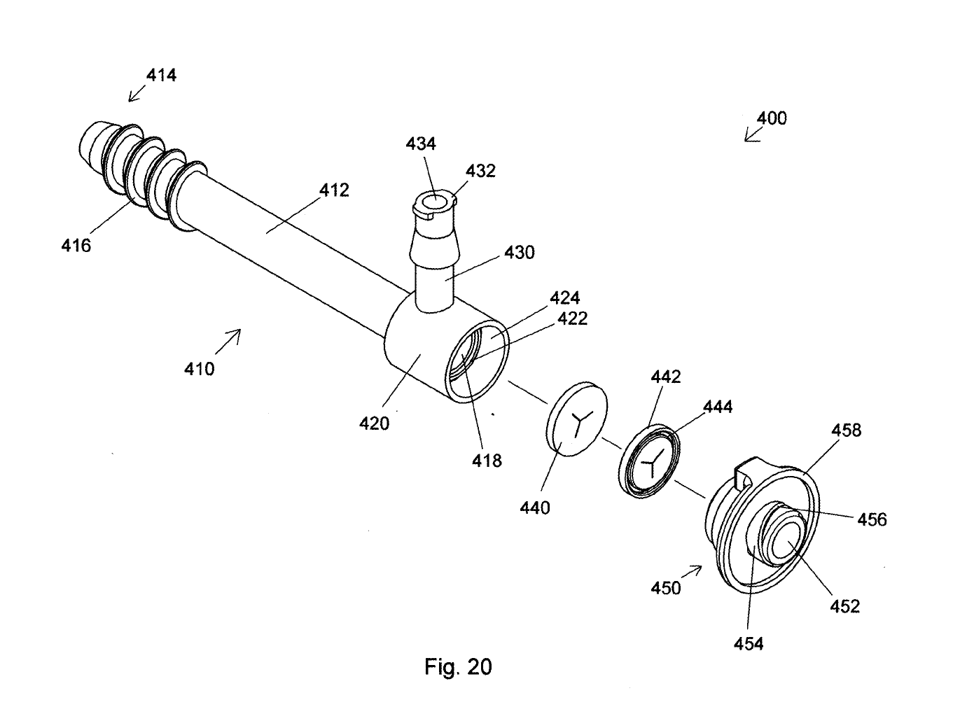

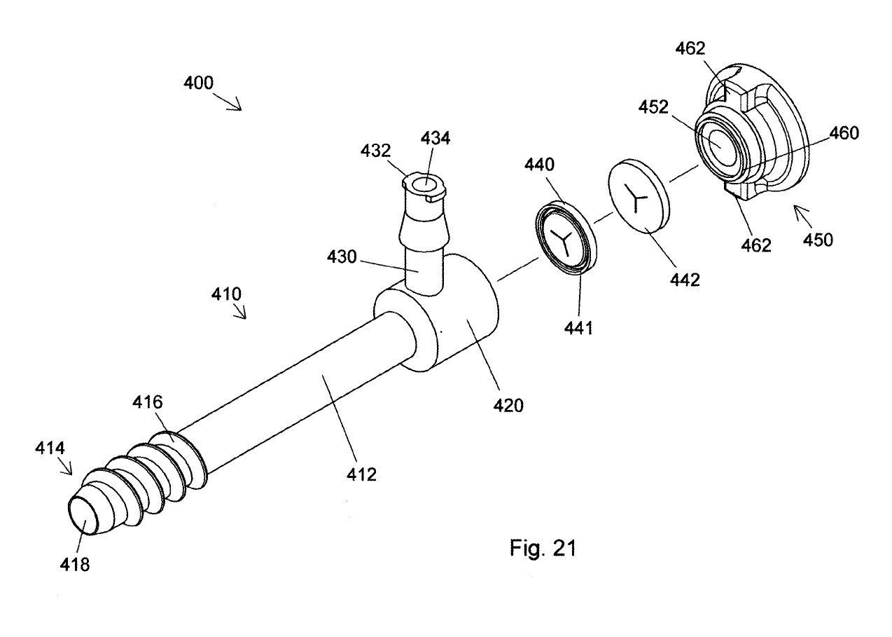

An alternate embodiment, the exploded assembly of which is depicted in FIGS. 20 and 21, functions in the same manner as sealing/stabilizing collar 300 and additionally functions as a cannula—an access device that prevents loss of irrigant from the intra-articular space during arthroscopic surgery. Sealing/stabilizing collar 400 is an assembly of distal element 410, distal sealing element 440, proximal sealing element 442, and proximal element 450. Sealing elements 440 and 442 are formed from a suitable elastomeric material and of a conventional design. As depicted, sealing elements 440 and 442 are both of a tricuspid design; however, alternate valve and flap designs readily apparent to those of skill in the art are contemplated and thus encompassed by the present invention. For example, in other illustrative embodiments, one of the seals may have an annular design. The particular design of the seals is not considered to be unique and thus does not form an integral part of the present invention.

Distal element 410 has an elongate tubular distal portion 412 with a distal end 414. As depicted herein, distal element 412 has threads formed adjacent to distal end 414. In other embodiments, threads 416 extend the entire length of distal portion 412. In still other embodiments threads 416 are not present. Proximal portion 420 of distal element 410 with proximal cylindrical recess 424 functions as a housing in which distal sealing element 440 and proximal sealing element 442 are positioned in the assembled device. Tubular inflow/outflow element 430 provides a fluid flow path to lumen 418 distal to sealing elements 440 and 442. Tubular inflow/outflow element with lumen 434 has formed at its distal end flange 432 configured for the removable mounting thereto of a Luer connector or Luer cap. Distal sealing element 440 is located and maintained in its position by groove 441 in the distal face of sealing element 440 coupling with annular rib 422 formed in the distal wall of proximal cylindrical recess 422 of distal element 410. Proximal element 450 is like sealing/stabilizing collar 300 in all aspects of form and function except as specifically hereafter described. The distal portion of proximal element 450 is configured for irremovable attachment to the proximal end of distal element 410. Distal facing annular rib 460 together with complementary groove 444 formed in the proximal face of proximal sealing element 442 locate and maintain the position of proximal sealing element 442 during assembly and use.

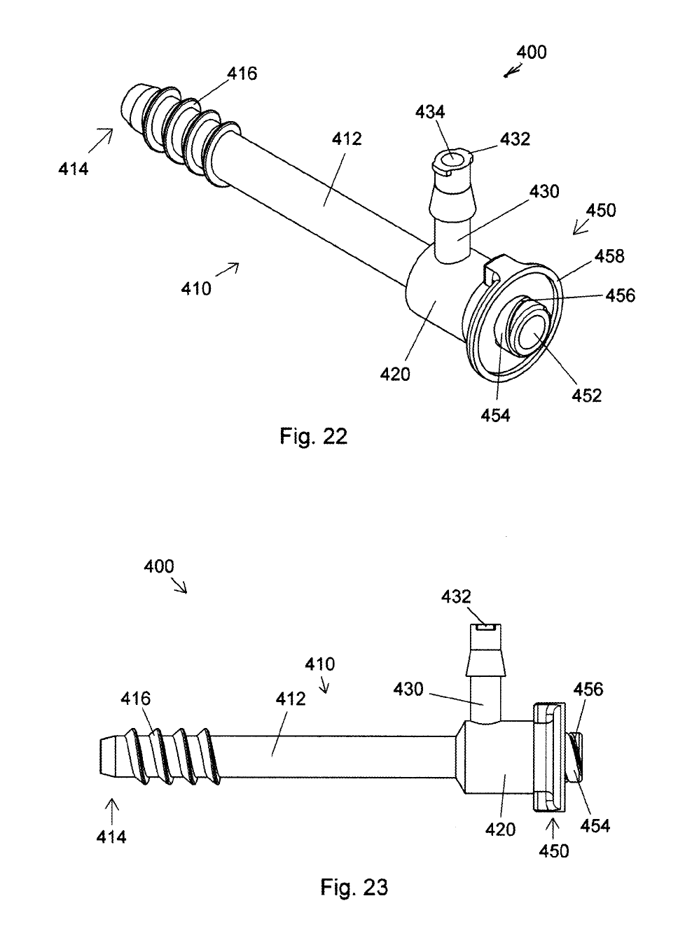

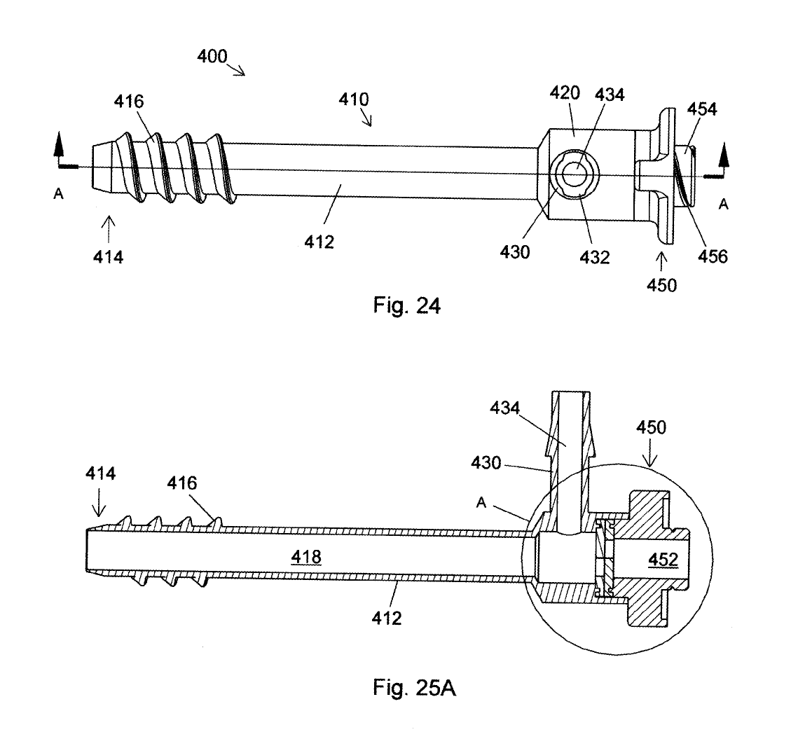

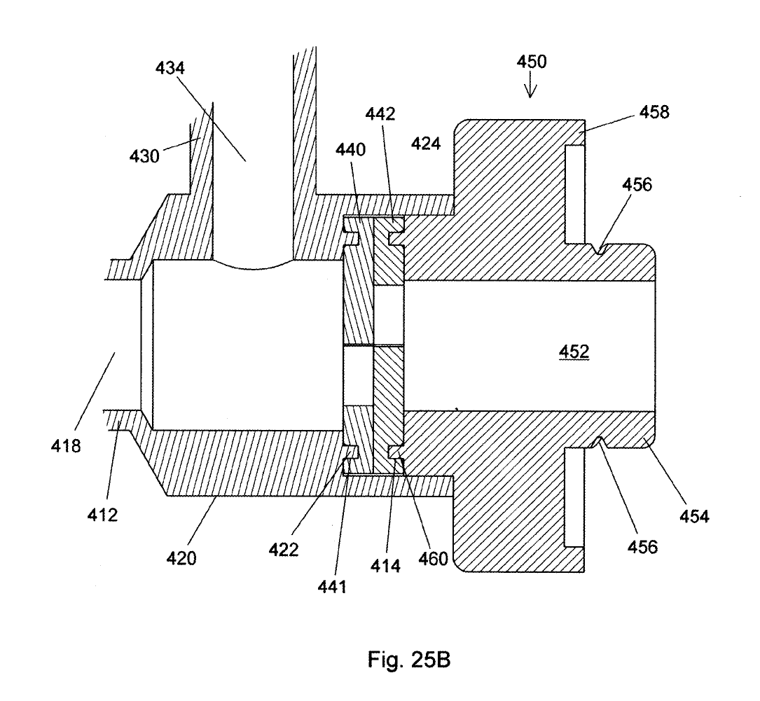

Assembled sealing/stabilizing collar 400 is depicted in FIGS. 22 through 25. As best seen in FIGS. 25A and 25B, distal sealing element 440 is maintained in its location by complementary rib 422 of distal element 410, and proximal sealing element 442 is maintained in its location by complementary rib 460 of proximal element 450. The proximal flow of fluid from the intra-articular space through lumen 452 of proximal element 450 is prevented by sealing elements 440 and 442. A fluid flow path is formed by lumen 418 of distal element 410 and lumen 434 of inflow/outflow tubular portion 430. Sealing/stabilizing collar 400 is removably mounted to shaver handpiece 200 in the same manner as sealing/stabilizing collar 300 and prevents vacuum leakage and stabilizes shaver 100 in handpiece 200 in the same manner. Additionally, sealing/stabilizing collar 400 may be inserted into the portal to the intra-articular space prior to insertion of shaver 100 into sealing/stabilizing collar 400 and the subsequent mounting of sealing/stabilizing collar 400 to shaver 100 in the manner previously described. The use of sealing/stabilizing collar 400 prevents the leakage of intra-articular fluid from the joint space when using shaver 100 or a similar conventional arthroscopy burr.

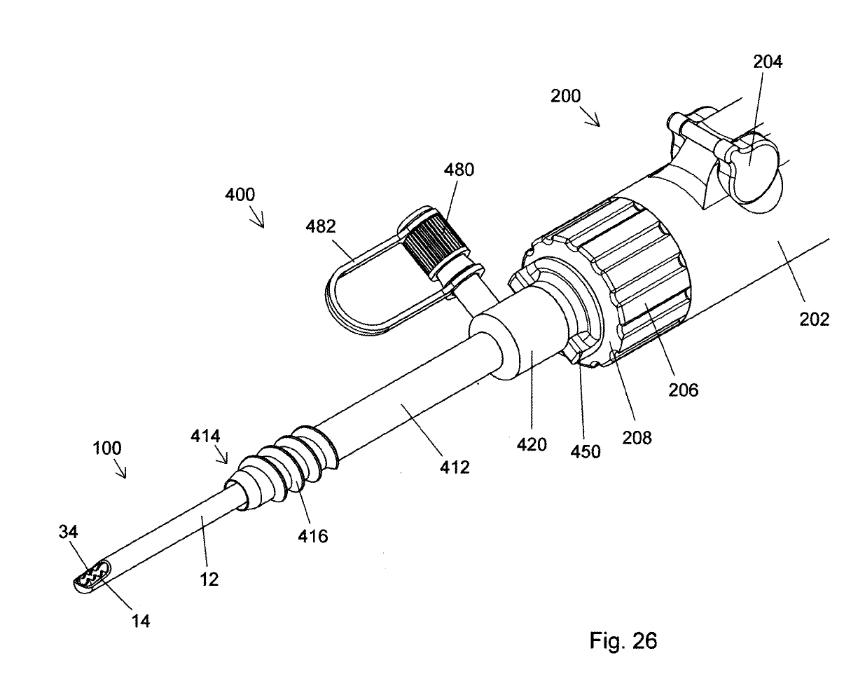

FIG. 26 depicts sealing/stabilizing collar 400 mounted to shaver 100 and handpiece 200 as during use. Luer cap 480, which may be removably attached to collar 400 by optional tether 482, prevents the outflow of fluid. Removing Luer cap 480 allows the outflow of fluid, or the inflow of fluid from an external source.

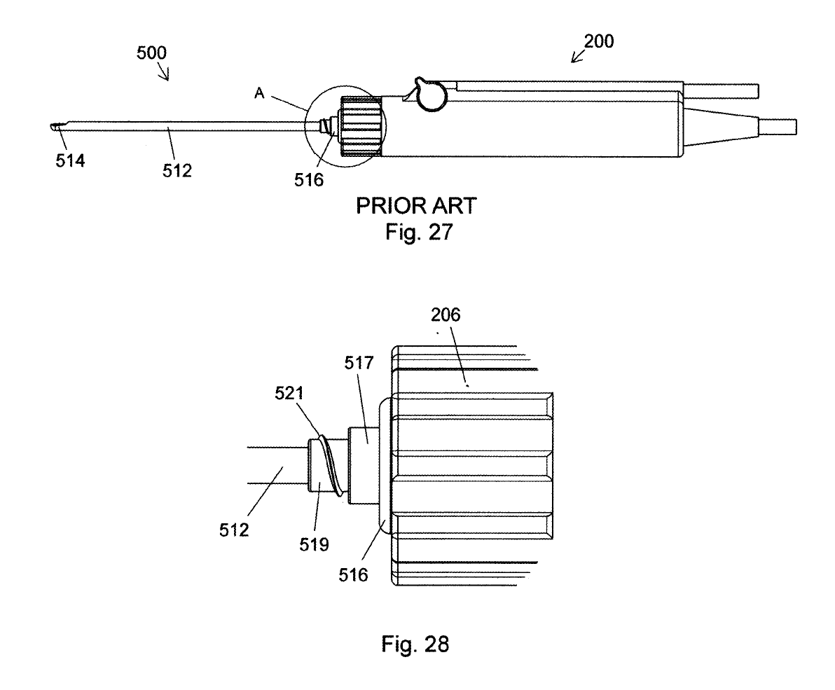

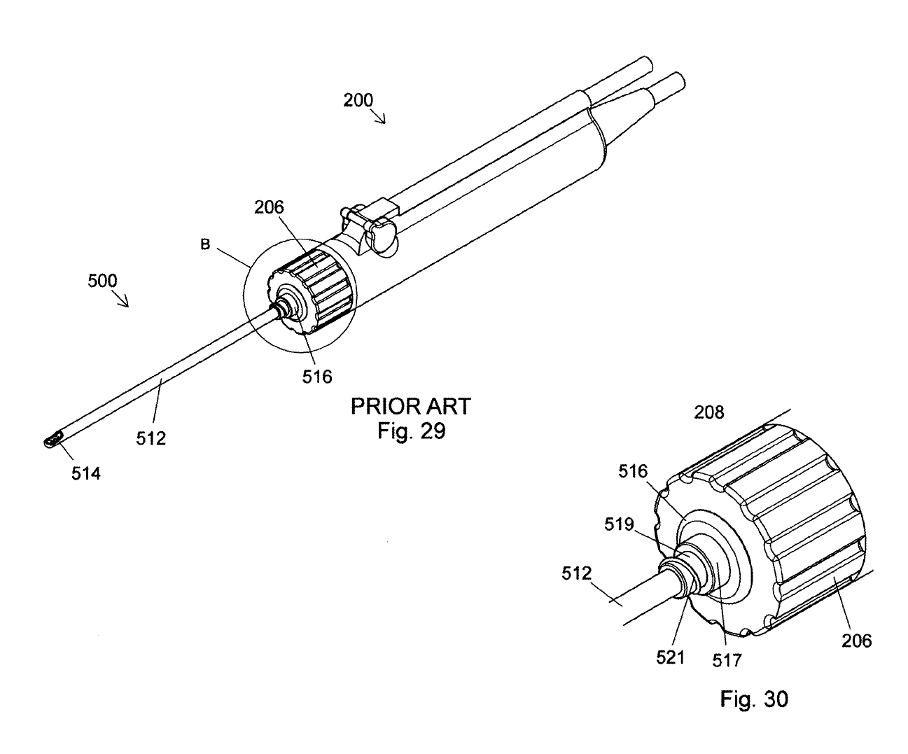

FIGS. 27 through 30 depicts an alternate prior art shaver 500 mounted in prior art shaver handpiece 200. Prior art shaver 500 is identical in all aspects of form and function to shaver 100 except as specifically subsequently described. Namely, outer hub 516 of shaver 500 does not have cylindrical distal recess 18 of hub 16 of shaver 100 as previously described and depicted in FIGS. 2 through 6. Rather, outer hub 516 of shaver 500 has a first cylindrical distal portion 517 and a second distal portion 519 on which are formed threads 521.

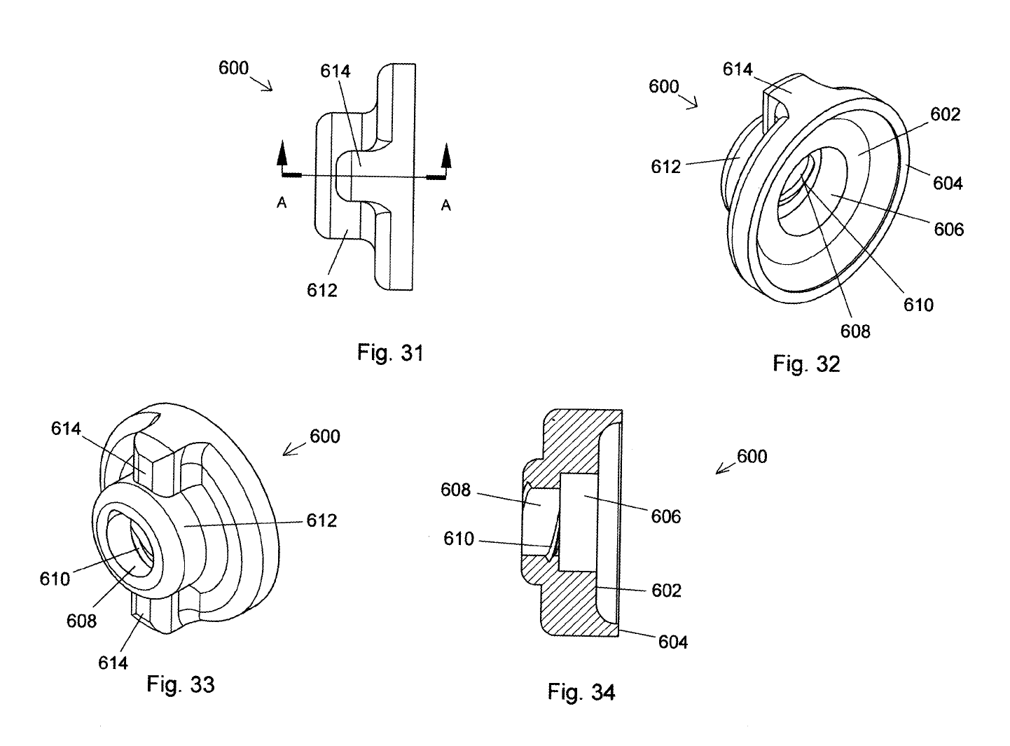

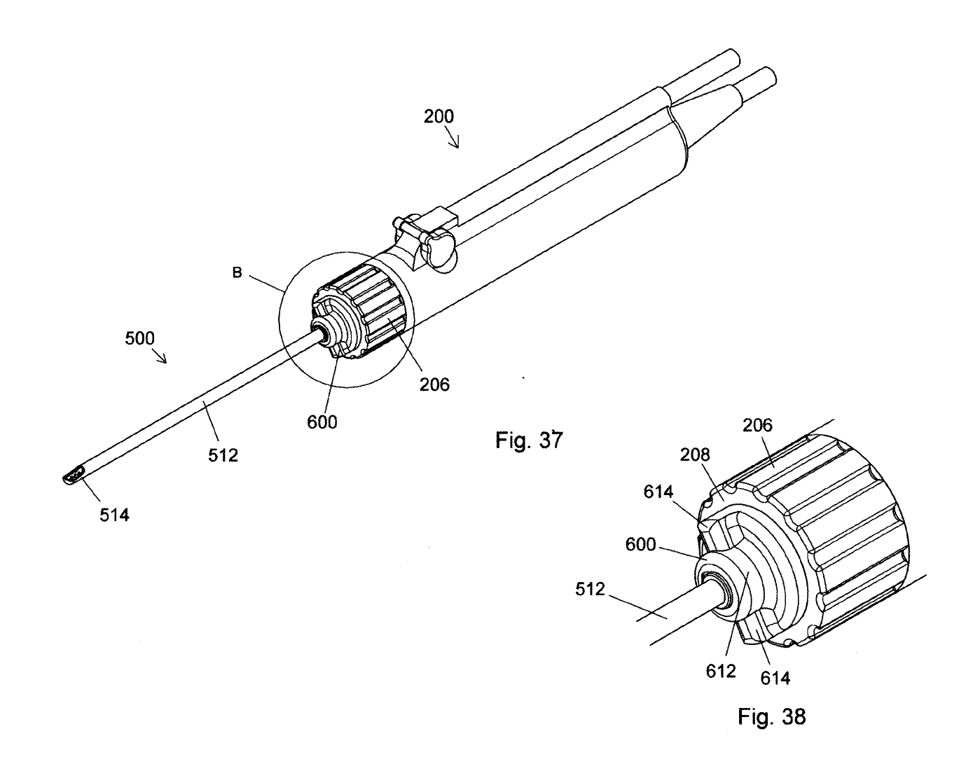

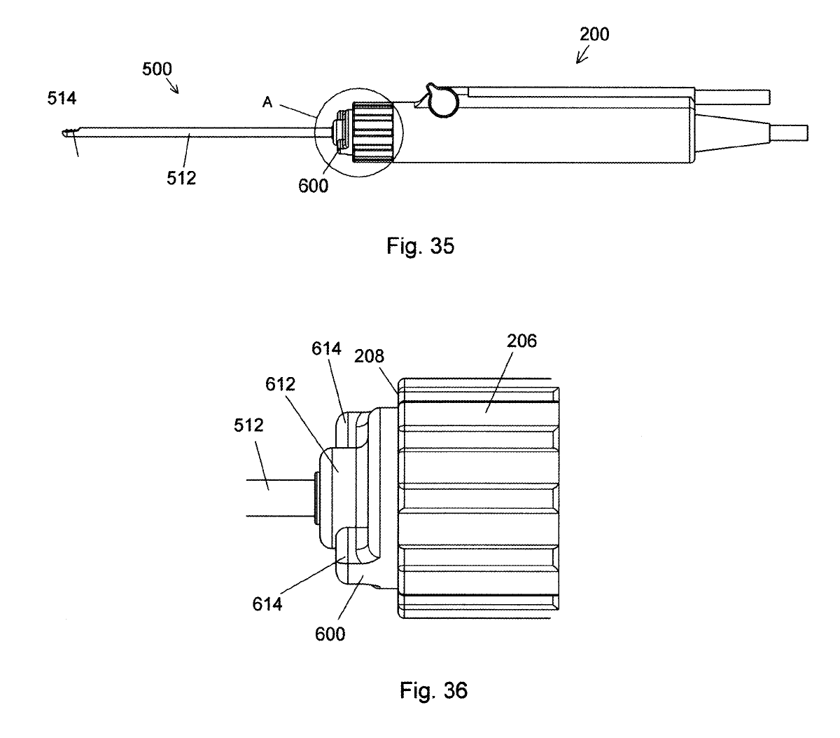

Shaver 500 may be stabilized relative to handpiece 200 in which it is mounted for use, and vacuum leakage prevented by mounting thereto alternate embodiment sealing/stabilizing collar 600 depicted in FIGS. 31 through 34. Sealing/stabilizing collar 600 is alike in form and function to sealing/stabilizing collar 300 in all aspects except as specifically subsequently described. Namely, sealing/stabilizing collar 600 does not have a cylindrical proximal portion 302 on which are formed threads 304 (FIGS. 11 through 15). Rather sealing/stabilizing collar 600 has a central lumen with a first cylindrical portion 606 configured to receive first cylindrical portion 517 of outer hub 516, and a second cylindrical portion 608 in which are formed threads 610. Second cylindrical portion 608 with threads 610 forms a fastener pair with second distal portion 519 with threads 521 of outer hub 516, allowing sealing/stabilizing collar 600 to be mounted to outer hub 517 thereby.

Sealing/stabilizing collar 600 is used in the same manner as sealing/stabilizing collar 300, and as depicted in FIGS. 36 through 38. After mounting shaver 500 in handpiece 200, sealing/stabilizing collar 600 is threaded onto second distal portion 519 of outer hub 517 until proximally extending rim 604 of sealing/stabilizing collar 600 is in firm contact with distal surface 208 of collet 206 of handpiece 200.

It will be understood that sealing/stabilizing collar 600 can be modified in the same manner as sealing/stabilizing collar 300 with the addition of an elongate distal tubular member and sealing elements to create a functionally equivalent device to collar 400 depicted in FIGS. 22 through 25.

### INDUSTRIAL APPLICABILITY

As noted previously, conventional endoscopic shavers tend to suffer from vacuum leakage which, in turn, undermines the efficiency of the device. In addition, the proximal connection between such devices and an associated powered handpiece has a tendency to be insufficiently rigid, whereby the longitudinal axis of the shaver device is permitted to flex and deflect from the desired linear position. Embodiments of the present invention address one or more of these disadvantages by providing a combination sealing and stabilizing collar component that both prevents vacuum leakage and ensures a rigid linear construction that may optionally be coupled to a conventional cannula for preventing intra-articular fluid leakage.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents.

What is claimed:

1. An endoscopic shaver assembly comprising:
a. an elongate tubular outer member characterized by a distal end having an outer cutting aperture formed therein, a proximal end that includes an outer hub configured for removable mounting within a cavity of an endoscopic shaver handpiece, and an elongate central lumen extending between said proximal and distal ends that defines the longitudinal axis of the shaver assembly, wherein the distal portion of said outer hub includes a first set of threads, further wherein distal axial movement of said outer member relative to said endoscopic shaver handpiece is precluded by means of an annular locking collet disposed about the outer periphery of said outer hub, further wherein said annular locking collet defines a gap between the outer periphery of said outer hub and an inner periphery of said collet;
b. an elongate tubular inner member characterized by a distal end having an inner cutting aperture formed therein, a proximal end that includes an inner hub configured for receiving torque from said endoscopic shaver handpiece and a proximal port for transmitting suction from said endoscopic shaver handpiece to said inner cutting aperture via an elongate central lumen that extends between said proximal and distal ends of said inner member, wherein said inner member is slidably received within the central lumen of said outer member and axially extended such that said inner and outer cutting apertures align; and
c. a combination sealing and stabilizing collar having a proximal end, a distal end, a central opening extending between said proximal and distal ends, a circumferential rim extending radially from said central opening characterized by proximally and distal facing surfaces, and a second set of threads complementary to and configured to engage with said first said of threads on said outer hub, wherein said combination sealing and stabilizing collar is mounted over the elongate outer member and positioned against the distal end of said outer hub, further wherein engaging said first and second sets of threads causes said proximal facing surface of said circumferential rim to be firmly seated against said annular locking collet and thereby seal the gap disposed between said outer hub and said annular locking collet and preclude lateral movement of outer member.

2. The endoscopic shaver assembly according to claim 1, wherein said first set of thread is disposed about the interior surface of a proximally extending recess provided the distal end of said outer hub and said complementary second set of threads is disposed about the exterior surface of a proximally facing cylindrical projection that extends from said central opening.

3. The endoscopic shaver assembly according to claim 1, wherein said first set of thread is disposed about the exterior surface of a distally facing cylindrical projection provided on the distal side of said outer hub and said complementary second set of threads is disposed about the interior surface of said central opening.

4. The endoscopic shaver assembly according to claim 1, wherein said distal facing surface of comprises one of more radially extending raised flanges that facilitate the threading of said first set of threads to said second set of threads.

5. The endoscopic shaver assembly according to claim 1, wherein said assembly further comprises a cannulated access device characterized by a central lumen into which said outer member is slidably received, further wherein the distal end of said combination sealing and stabilizing collar threadedly connected to the proximal end of said cannulated access device.

6. The endoscopic shaver assembly according to claim 5, wherein the central lumen of said cannulated access device comprises one or more sealing elements.

7. The endoscopic shaver assembly according to claim 6, wherein at least of one of said one or more sealing elements comprises a flap valve.

8. The endoscopic shaver assembly according to claim 5, wherein said cannulated access device further comprises a tubular inflow/outflow channel normal to and in fluid communication with the central lumen of said cannulated access device.

9. The endoscopic shaver assembly according to claim 5, wherein the exterior surface of the distal end of said cannulated access device is provided with a spiral thread that facilitates atraumatic tissue insertion.
